(19) United States
(12) Patent Application Publication
(10) Pub. No.: US 2011/0029063 A1
(43) Pub. Date: Feb. 3, 2011

(54) AUXETIC STENTS

(75) Inventors: Zheng-Dong Ma, Dexter, MI (US); Yuanyuan Liu, Ann Arbor, MI (US)

Correspondence Address: GIFFORD, KRASS, SPRINKLE,ANDERSON & CITKOWSKI, P.C, PO BOX 7021, TROY, MI, 48007-7021, US

(73) Assignee: MKP Structural Design Associates, Inc.

(21) Appl. No.: 12/829,815

(22) Filed: Jul. 2, 2010

Related U.S. Application Data
(63) Continuation-in-part of application No. 12/267,867, filed on Nov. 10, 2008.

(51) Int. Cl. A61F 2/82

(52) U.S. Cl. 623/1.16

(57) ABSTRACT

Stents of the type used to treat and prevent localized flow constriction in body vessels are based upon negative Poisson's ratio (NPR) structures. An auxetic stent constructed in accordance with this invention comprises a tubular structure having two ends defining a length with a central longitudinal axis and an axial view defining a cross section. The tubular structure is composed of a plurality of unit cells with two different configurations, called V-type and X-type. In V-type auxetic stents, each unit cell comprises a pair of side points A and B defining a width, a first pair of members interconnecting points A and B and intersecting at a point C forming a first V shape, and a second pair of members interconnecting points A and B and intersecting at a point D forming a second V shape. In X-type auxetic stents, each unit cell comprises eight points from A to H defining an outline of the unit cell. Eight straight or curved members interconnecting points A and B, B and C, C and D, C and E, E and F, F and G, G and H, G and A, respectively, forming the X-type unit cell. In both configurations, the unit cells are connected in rows and columns, such that compression of the structure between the two ends thereof causes the cross section of the structure to shrink in size. The auxetic structure configurations invented can also be used, with similar dimensions or significantly different dimensions, for other applications, such as in a nano-structural device, a tubal fastener design, or in an application associated with a large oil pipe or other pipelines.

### REFERENCE TO RELATED APPLICATION

This application is a continuation-in-part of U.S. patent application Ser. No. 12/267,867, filed Nov. 10, 2008, the entire content of which is incorporated herein by reference.

### FIELD OF THE INVENTION

This invention relates generally to medical/surgical stents and, in particular, to stents based upon negative Poisson's ratio (NPR) structures.

### BACKGROUND OF THE INVENTION

Vascular stenting dates back to the late 1970s and the use of angioplasty balloons to treat vessel constrictions. Although arteries could be opened successfully using a balloon, in some cases the vessel would collapse after the balloon was deflated. Another problem has been restinosis or reblocking. Approximately 30 percent of all coronary arteries began to close up again after balloon angioplasty. Bypass and graft surgeries and miniaturized tools delivered via catheter were all used to find solutions.

Stents were developed in the 1980s. A stent is a metal (or other material) tube or scaffold that is inserted after balloon angioplasty. The device is mounted on a balloon and opened inside the vessel. In 1994 the first (Palmaz-Schatz) stent was approved for use in the U.S. Over the next decade, several generations of bare metal stents were developed, with each succeeding one being more flexible and easier to deliver.

Although the rates were reduced, bare metal stents still experienced reblocking (typically at six-months) in about 25 percent of cases, necessitating a repeat procedure. It was discovered that restenosis, rather than being a recurrence of coronary artery disease, was actually due to the growth of smooth muscle cells, analogous to scarring in the vicinity of the angioplasty site. A variety of drugs were tested to interrupt the biological processes that caused restenosis. Clinical trials began with stents that were coated with these drugs, sometimes imbedded in a thin polymer for time-release.

While drug-eluting stents have been very successful in reducing restenosis, other factors remain important in stent choice and placement. These considerations include correct sizing of the stent diameter and length to match the characteristics of the lesion, or blocked area. It is also critical that the stent is expanded fully to the arterial wall, since under-expansion can lead to blood clots, or Sub-Acute Thrombosis (SAT).

Poisson's ratio (v), named after Simeon Poisson, is the ratio of the relative contraction strain, or transverse strain (normal to the applied load), divided by the relative extension strain, or axial strain (in the direction of the applied load). Some materials, called auxetic materials, have a negative Poisson's ratio (NPR). If such materials are stretched (or compressed) in one direction, they become thicker (or thinner) in perpendicular directions.

The vast majority of auxetic structures are polymer foams. U.S. Pat. No. 4,668,557, for example, discloses an open cell foam structure that has a negative Poisson's ratio. The structure can be created by triaxially compressing a conventional open-cell foam material and heating the compressed structure beyond the softening point to produce a permanent deformation in the structure of the material. The structure thus produced has cells whose ribs protrude into the cell resulting in unique properties for materials of this type.

Published U.S. Patent Application Serial No. 2006/0129227, entitled “Auxetic Tubular Liners,” uses a geometry of inverted hexagons in order to affect auxetic properties in a tubular structure. These inverted hexagons are not regular hexagons, and instead essentially comprise a hexagon having first and second sides opposite and parallel to one another, and then third, fourth, fifth and sixth inwardly-inclined sides joining them. The inverted hexagons may also be linked together via the vertices of their first and second sides, although this may result in non-auxetic regions while still retaining an overall auxetic structure. The first and second sides may also be replaced with sides having relatively inflexible branched sections. Thus, for example first and second sides can be replaced with a first side having first and second vertices, and with first and second arms extending from each of the first and second vertices, each of the first and second arms making an internal angle with the first side of between 90 and 180 degrees. For example, internal angles of between 91 and 179 degrees can be made, e.g. 125, 130, 135, 140, 145 or 150 degrees. Third, fourth, fifth and sixth sides can then depend from the first and second arms of the first and second sides, thus completing the polygons.

While the auxetic structures just described may find application in medical/surgical stenting, the devices present various deficiencies. Firstly, the structures rely upon an NPR “material” with very small scale unit cell. As a result, a very large number of unit cells are used, limiting the tubular liners to round shape of cross-section. Second, the structures are limited to folded two-dimensional designs, precluding true three-dimensional shapes. Additionally, the disclosed NPR structures provide only for a homogeneous distribution of the unit cell. It would be more advantageous to allow for varied unit cell structure as part of a ‘hybrid’ structure that can be functionally designed with respect to the requirements in various applications.

### SUMMARY OF THE INVENTION

This invention relates generally to stents of the type used to treat and prevent localized flow constriction in body vessels and, in particular, to stents based upon negative Poisson's ratio (NPR) structures.

An auxetic stent constructed in accordance with this invention comprises a tubular structure having two ends defining a length with a central longitudinal axis and an axial view defining a cross section. The tubular structure is composed of a plurality of unit cells. Each unit cell comprises a pair of side points A and B defining a width, a first pair of members interconnecting points A and B and intersecting at a point C forming a first V shape, and a second pair of members interconnecting points A and B and intersecting at a point D forming a second V shape. The unit cells are connected in rows, with the point B of one cell being connected to point A of an adjoining cell until completing a band around the tubular structure. The unit cells are further connected in columns along the length of the tubular structure with the point D of one cell being connected to point C of an adjoining cell until spanning the length of the tubular structure. Compression of the structure between the two ends thereof causes the cross section of the structure to shrink in size.

In certain preferred embodiments, the members define straight segments, and the cross section defines a regular polygon, such as a square, hexagon, octagon, decagon, dodecagon, or any higher-order polygon. Alternatively the members are curved, in which case the cross section of the tubular structure defines a circle. One advantage of the invention is that different types of unit cells may be combined to form hybrid structure. Such an approach may be particularly advantageous in terms of medical/surgical stent designs in that particular portions of the length of the stent such as the mid section may have a larger girth and/or be more resistant to externally applied pressure.

### BRIEF DESCRIPTION OF THE DRAWINGS

FIG. 1 shows a basic V-type cell according to the present invention;

FIG. 2 shows an array of the V-type auxetic stent cells assembled in a flat plane prior to tubular fabrication;

FIG. 3 illustrates a negative Poisson ratio effect in conjunction with an array of V-type unit cells;

FIG. 4A is a front view of a honeycomb constructed utilizing a V-type cell;

FIG. 4B is a through-hole (side) view of a honeycomb constructed utilizing the V-type cell;

FIG. 5 illustrates an unfolding mechanism of a medical/surgical stent constructed with V-type auxetic cells;

FIG. 6 is a drawing of a different unit cell (X-type) constructed in accordance with the invention;

FIG. 7 shows a resulting X or “bowtie” shape that may be arranged in an array of interconnecting unit cells;

FIG. 8 illustrates a negative Poisson ratio effect in conjunction with an array of X-type unit cells;

FIG. 9A is a drawing of an X-type auxetic stent constructed from X-type unit cells;

FIG. 9B shows a twelve-sided cross-sectional geometry;

FIGS. 10A to 10C is a series of drawings which shows the unfolding mechanism of an X-type stent having a six-sided cross section as opposed to a twelve-sided cross section of FIG. 9B;

FIG. 10A shows the stent in the compressed or folded mode;

FIG. 10B shows the stent in an intermediate unfolded state;

FIG. 10C shows the stent in a fully expanded configuration;

FIG. 11A shows a square V-type auxetic stent from an end-side view;

FIG. 11B shows a hexagonal or honeycomb V-type auxetic stent from an end-side view;

FIG. 11C shows an octagon V-type auxetic stent from an end-side view;

FIG. 11D shows a dodecagon V-type auxetic stent from an end-side view;

FIG. 12A is a top view of a honeycomb V-type auxetic stent;

FIG. 12B is an end-side view of a honeycomb V-type auxetic stent;

FIG. 12C is an expanded plane view of a honeycomb V-type auxetic stent;

FIG. 12D is an isometric view of a honeycomb V-type auxetic stent

FIG. 13A illustrates a different honeycomb V-type auxetic stent design showing a side view;

FIG. 13B illustrates a different honeycomb V-type auxetic stent design showing an end-side view;

FIG. 13C illustrates a different honeycomb V-type auxetic stent design showing a top view;

FIG. 13D illustrates a different honeycomb V-type auxetic stent design showing a an isometric view;

FIG. 14 shows an octagonal V-type auxetic stem with bent tendon and stuffer members;

FIG. 15 illustrates a different configuration of an octagonal V-type auxetic stent constructed in accordance with the invention;

FIG. 16 shows an auxetic stent with a cross section featuring a ten-sided arrangement;

FIG. 17A is a front view of a 7-cell honeycomb V-type auxetic stent design;

FIG. 17B is an end-side view of a 7-cell honeycomb V-type auxetic stent design;

FIG. 17C is a top view of the 7-cell honeycomb V-type auxetic stent design;

FIG. 17D is an isometric view of the 7-cell honeycomb V-type auxetic stent design;

FIG. 18A is a front view of an octagonal X-type auxetic stent configuration;

FIG. 18B is an end-side view of the octagonal X-type auxetic stent configuration FIG. 18C shows a stent having a isometric view;

FIG. 19A shows a front view of higher-order X-type auxetic structure having a twelve-sided cross section;

FIG. 19B shows an end-side view of a higher-order X-type auxetic structure;

FIG. 19C shows an expanded plane view of a higher-order X-type auxetic structure;

FIG. 19D shows an isometric representation of a higher-order X-type auxetic structure;

FIG. 20A is an expanded plane view of a cubic V-type auxetic stent;

FIG. 20B is a top view of a cubic V-type auxetic stent;

FIG. 20C is a four-sided cross section of a cubic V-type auxetic stent;

FIG. 20D is an isometric view of a cubic V-type auxetic stent;

FIG. 21 illustrates a square X-type auxetic stent constructed in accordance with the present invention;

FIG. 22A illustrates an example configuration of the hybrid stents that combine V-type and X-type unit cells together: left half is made of V-type cells and right half is made of X-type cells;

FIG. 22B illustrates second example configuration of the hybrid stents that combine V-type and X-type unit cells together: middle section is made of X-type cells, and the rest part is made of V-type cells;

FIG. 22C illustrates third example configuration of the hybrid stents that combine V-type and X-type unit cells together: middle section is made of V-type cells, and the rest part is made of X-type cells;

FIG. 22D illustrates another example configuration of the hybrid stents that combine V-type and X-type unit cells together: left, middle, and right sections are made of X-type cells and the rest part is made of V-type cells;

FIG. 23 illustrates an example configuration of the stents designed to have a bulging effect by varying cell variables defined in FIG. 6 along the length of the stent;

FIG. 24 illustrates computer simulation results of the stent shown in FIG. 23, which shows a bulge is formed in the middle of the stent along with elongation of the stent under a tension force;

FIG. 25 illustrates an example configuration of the stents designed to have a bulging effect at the two ends of the stent by varying cell variables defined in FIG. 6 along the length of the stent;

FIG. 26 illustrates computer simulation results of the stent shown in FIG. 25, which shows two bulges are formed at the ends of the stent along with elongation of the stent under a tension force;

FIGS. 27A and 27B illustrate example configurations of the stents that can deform to a shape with predefined curvature by varying cell variables defined in FIG. 6 along the circle of the stent; and

FIG. 28 illustrates computer simulation results of the stent shown in FIG. 27B, which shows the stent deformed to a curved shape along with elongation of the stent under a tension force.

### DETAILED DESCRIPTION OF THE INVENTION

This invention is directed to negative Poisson's ratio (NPR) or auxetic structures and, in particular, to three-dimensional auxetic medical/surgical stents. As an introduction, co-pending U.S. patent application Ser. No. 12/267,867, describes a pyramid-shaped unit cell having four base points defining the corners of a square lying in a horizontal plane. Four “stuffer” members of equal length extend from a respective one of the base points to a fifth point spaced apart from the plane. Four “tendon” members of equal length, but shorter than the stuffers, extend from a respective one of the base points to a sixth point between the fifth point and the plane. In a preferred embodiment, a line drawn through the fifth and sixth points is normal to the plane.

The stuffer and tendon members may have a rectangular, round, or other cross section. The stuffers and the tendons may be of equal or unequal length, and may have equal or unequal cross sections. The stuffer and tendon members in a stent may be made of same material or different materials for the stuffers and tendons. The stuffers and tendons may be made of biocompatible metals (e.g., stainless steels, gold-plated hybrid materials, titanium alloys), polymers (including biodegradable and bioabsorbable polymers), shape memory alloys or polymers, fibers, fiber ropes, or other materials. In general case, stuffers should be designed for carrying compressive load, and tendons tensile load. However, depending on the application, stuffers can also carry tensile load and tendon compressive load. They can also switch their roles in an application. All of these should be carefully considered when designing an optimum stent. In one preferred embodiment, the stuffers and tendons are made of stainless steel, with the cross-sectional area of the tendons being less than the cross-sectional area of the stuffers. The geometry, dimensions or composition of the tendons or stuffers may be varied to achieve different effective material properties along different directions, to achieve a different effective Young's modulus along different directions, or to achieve different effective Poisson's ratios along different directions. The structures may achieve different material densities in different layers.

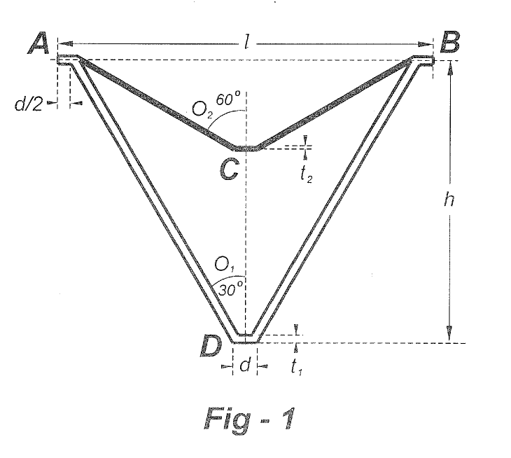

One stent structure according to this invention utilizes a two-dimensional portion of the pyramid-shaped unit cell described in the co-pending application just discussed. FIG. 1 shows a basic V-type cell according to the invention. The cell comprises two “stuffer” members 102, 102′ and two “tendon” members 104, 104′. Members 102 interconnect at a bottom point 110 and members 104 interconnect at a point 114 above the point 110 in the figure. The upper points of both the members 102, 104 connect on either side at points 112, 112′. In the preferred embodiment, a line 106 drawn through points 112, 114 is perpendicular to a line 108 drawn through points 112, 112′.

The angle O₁ between each member 102, 102′ and the vertical center line 106 may be on the order of 30 degrees, whereas the angle O₂ between each member 104, 104′ and the vertical center line 106 may be on the order of 60 degrees. While these two angles are preferred in some embodiments, other angles may be used, resulting in an auxetic structure of the type described herein, so long as members 102, 102′ are longer than members 104, 104′.

Points 112, 112′ are spaced apart at a distance “l” and may include small land areas in the event that spot welding or other such processes are used for interconnection. The height of the cell is defined as “h”. The points 110, 114 where the members 102, 104 interconnect along line 106 may also include small flat portions or lands, again, for spot-welding or other purposes. If the structure is made from a unitary piece of material with removal carried out through laser etching or other such processes, the lands or flat portions just described may or may not be eliminated. The members 102, 104 may be straight or curved and of any suitable cross section, with constant thicknesses t₁ and t₂ which may be the same or different, or variable thicknesses t₁ and t₂, which are varied along the axial direction. For use as a medical/surgical stent, the V-type auxetic cell just described may have a length l equal to 10 mm or thereabouts, a height h equal to 8 mm or thereabouts, depending upon the application and which configuration is used. The auxetic structure configuration invented here can also be used, with similar dimensions or significantly different dimensions, for other applications, such as in a nano-structural device, a tubal fastener design, or in an application associated with a large oil pipe or other pipelines.

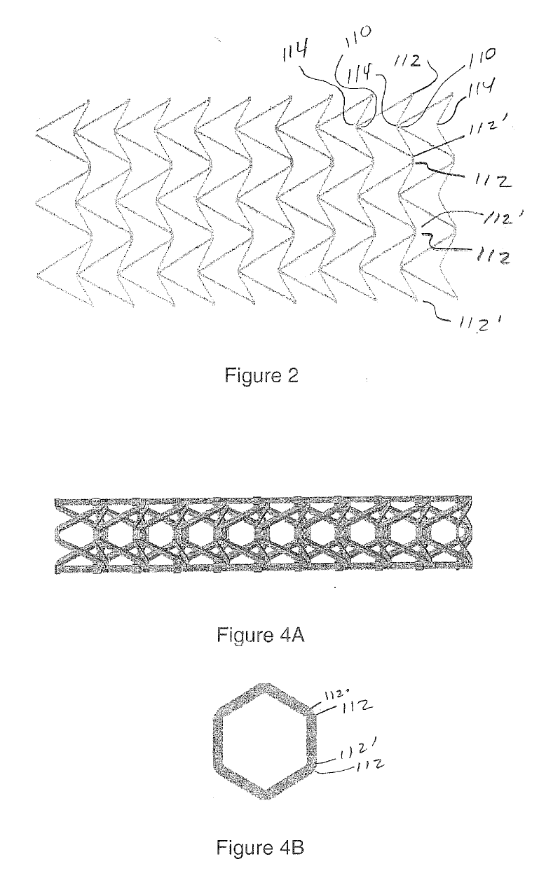

FIG. 2 is a drawing which shows an array of the V-type auxetic stent cells assembled in a flat plane prior to tubular fabrication. As can be seen from this drawing, to fashion the array, points 112′ are connected to adjoining points 112 and lower points 110 are connected to upper points 114 of cells above and below one another, creating a substantially regular pattern. The negative Poisson ratio effect is illustrated in FIG. 3. As the structure of the type shown in FIG. 2 is compressed, the array shrinks overall and, with further compression, becomes even smaller as shown in the right-most illustration in the figure.

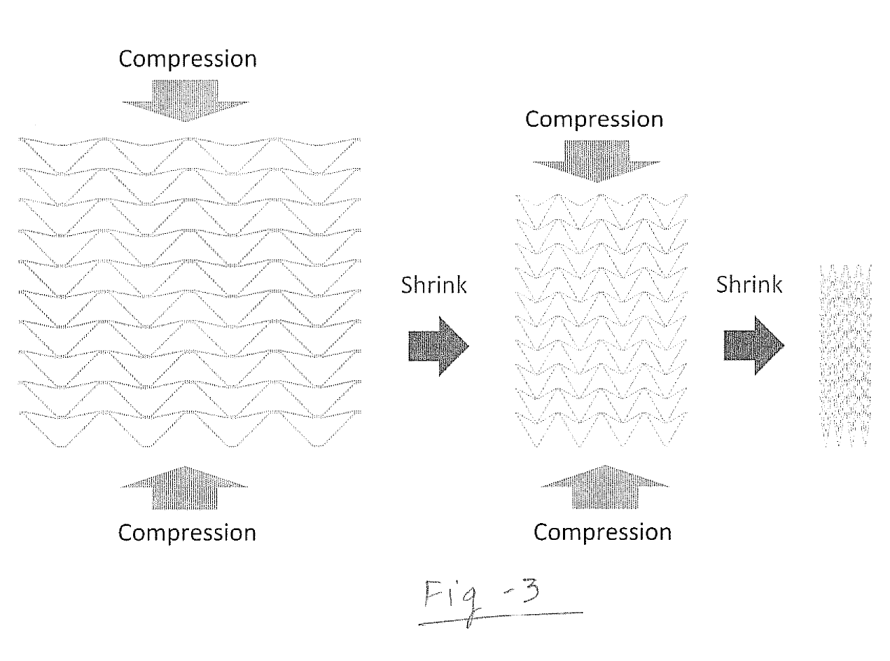

FIG. 4A is a front view drawing of a honeycomb of said extent constructed utilizing the V-type cell. In this case, referring to the through-hole side view of FIG. 4B, three of the V cells are interconnected with points 112 connecting to points 112′ in each case, resulting in a honeycomb cross section. Continuing the reference to FIG. 2, when the structure of FIG. 4A is compressed from end to end, the overall stent shrinks, as shown schematically in FIG. 3. (with four of the V cells and a hexagonal cross section)

FIG. 5 illustrates an unfolding mechanism of a medical/surgical stent constructed with V-type auxetic cells shown in FIG. 3. FIG. 5A shows the stent in a compressed or folded mode. FIG. 5B shows an intermediate unfolded state, with the ends of the stent and cross section of the stent expanding, and FIG. 5C shows the stent in a final expanded mode.

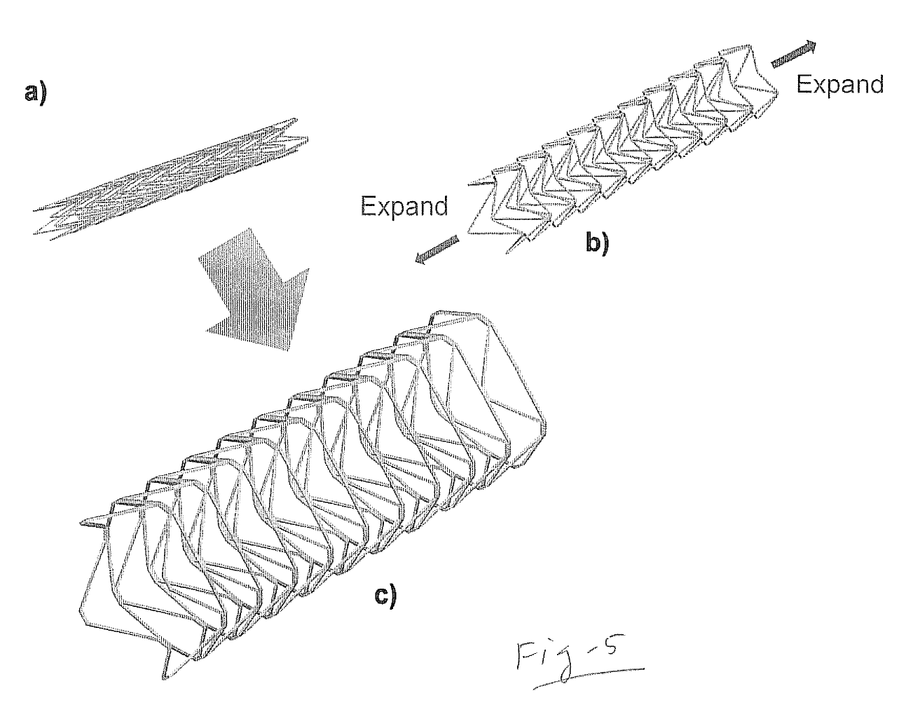

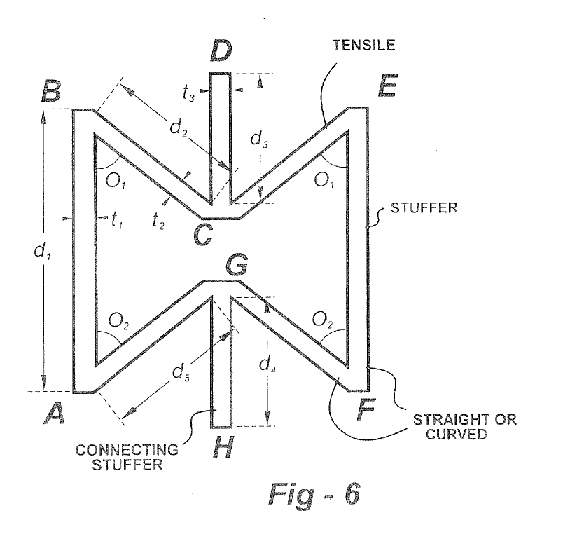

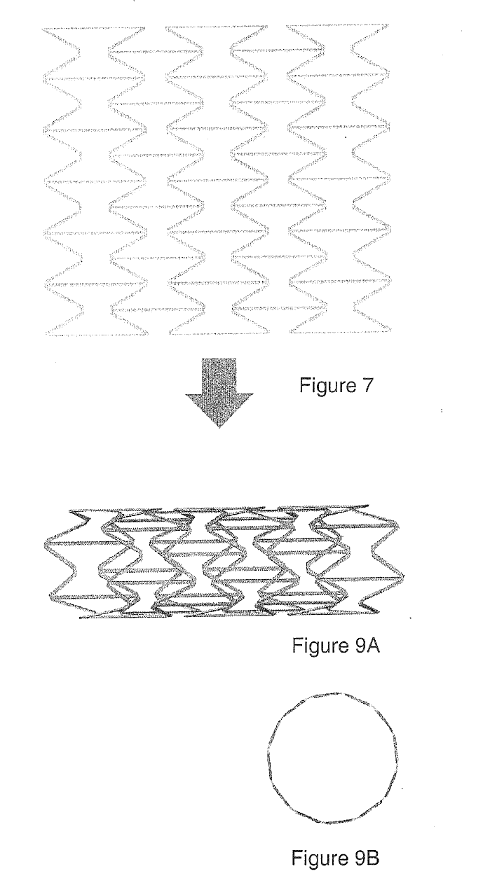

FIG. 6 is a drawing of a different unit cell constructed in accordance with the invention, in this case an X-type cell. The various component parts are defined as follows. d₁ is the height of the stuffers of the unit cell; d₂, is the length of the top “tensile” members; d₃ is the height of the top connecting stuffer member; d₄ is the height of the bottom connecting stuffer member; d₅ is the length of the bottom “tensile” member; t₁ represents the half thickness of the stuffers; t₂ represents the thickness of the tensile members; O₁ is the angle between a top tensile member and the vertical line; and O₂ is the angle between a bottom tensile member and the vertical line. This resulting X or “bowtie” shape also may be arranged in an array of interconnecting unit cells, as shown in FIG. 7, and compressed as shown in FIG. 8, to shrink in X and Y directions through the application of compressive force. As with the V-type cell, a two-dimensional array of the X-type cells may be wrapped around one another and shaped into a tube shape, resulting into an X-type auxetic stent shown in FIG. 9A. Again, as with the V-type cell, different numbers of unit cells may be arranged in this way, resulting in cross sections with different geometries. In FIG. 9B, a twelve-sided cross-sectional geometry is achieved. Furthermore, as with the V-type cell, the auxetic structure configuration invented here can also be used, with similar dimensions or significantly different dimensions, for other applications, such as in a nano-structural device, a tubal fastener design, or in an application associated with a large oil pipe or other pipelines.

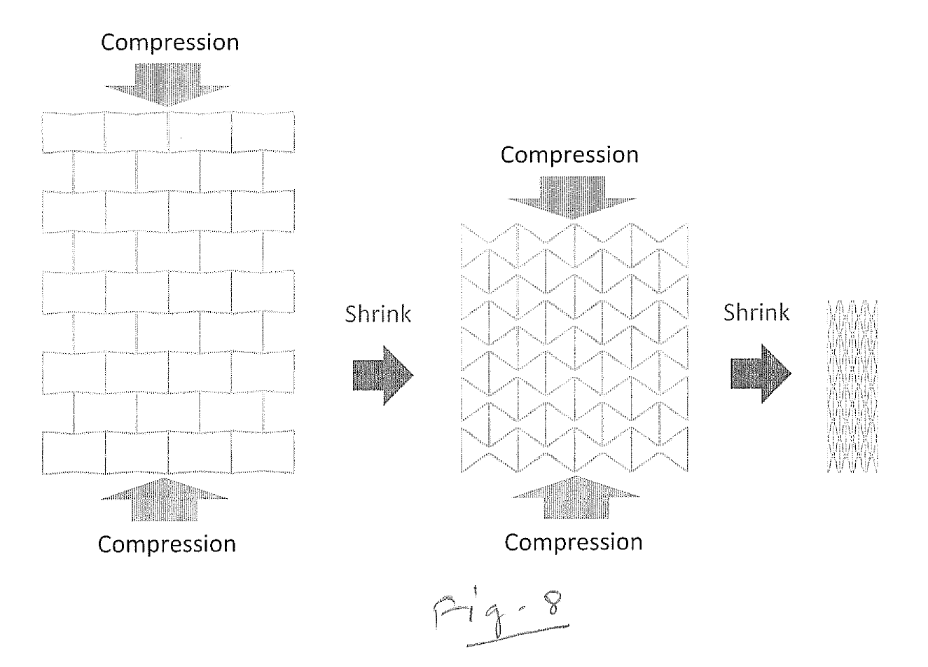

FIG. 10 is a series of drawings which shows the unfolding mechanism of an X-type stent shown in FIG. 8 having an eight-sided cross section as opposed to a twelve-sided cross section of FIG. 9B. FIG. 10A shows the stent in the compressed or folded mode; FIG. 10B shows the stent in an intermediate unfolded state; and FIG. 10C shows the stent in a fully expanded configuration.

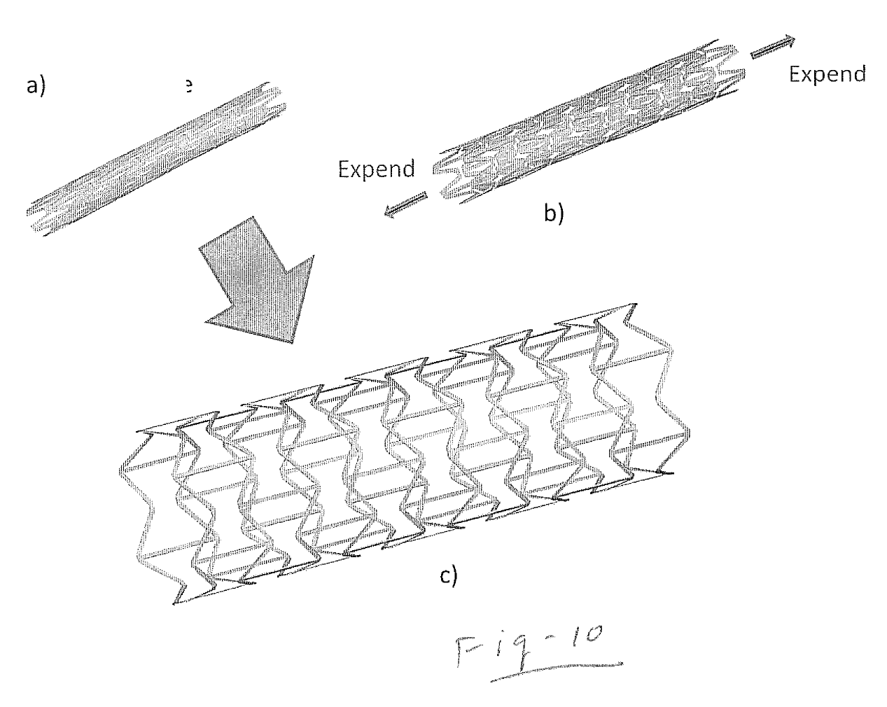

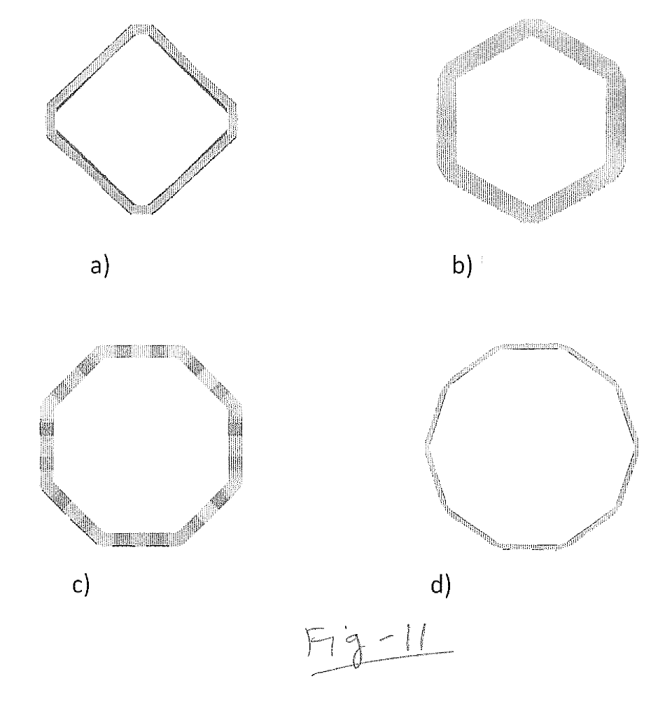

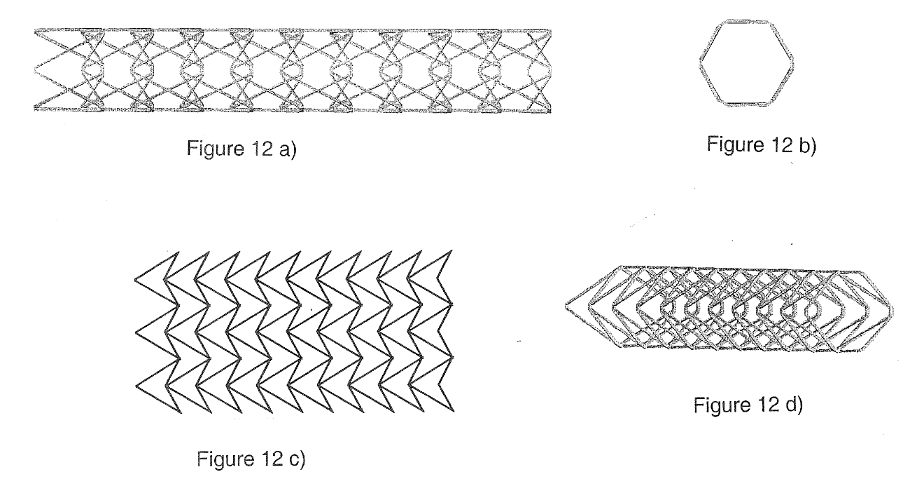

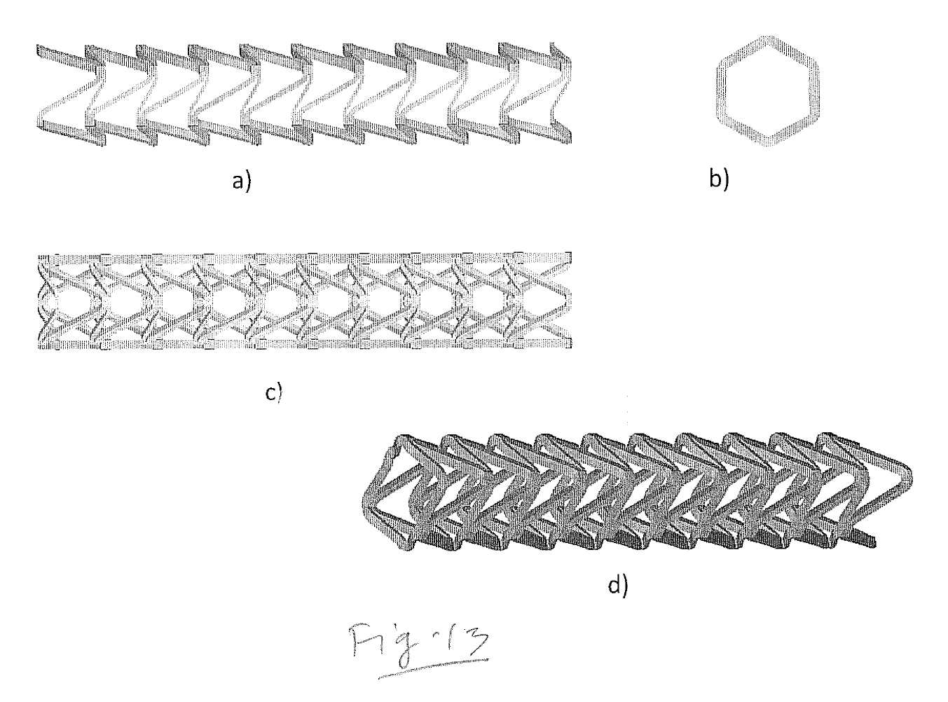

As discussed, auxetic stents constructed in accordance with this invention may have various cross-sectional geometries. As one example, FIG. 11 shows four stents with different cross-sections, including the square shape of FIG. 11A; the hexagonal or “honeycomb” shape of FIG. 11B, the octagonal shape of FIG. 11C, and the ten-sided polygon (decagon) of FIG. 11D. FIG. 12 shows a stent with a hexagonal or honeycomb V-type auxetic stent from a front view (FIG. 12A), an end-side view (FIG. 12B), a partially expanded plane view (FIG. 12C) and an isometric view (FIG. 12D). FIG. 13 illustrates a different honeycomb V-type auxetic stent design, wherein three unit cells are used around the circumference of the device, with the tendon and stuffer members being bent so that a hexagonal cross section is shown as illustrated in FIG. 13B. FIG. 13A is a front view, FIG. 13C is a top view, and FIG. 13D is an isometric view of this design.

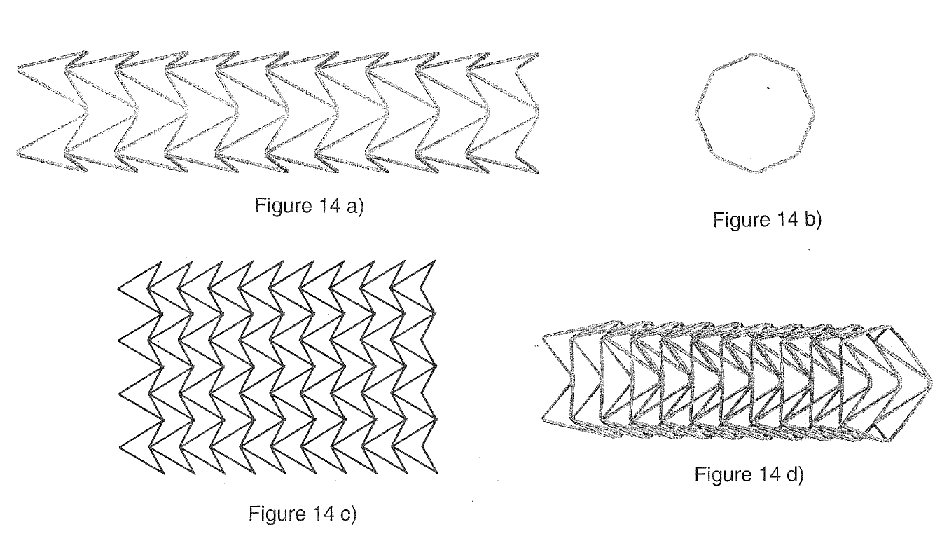

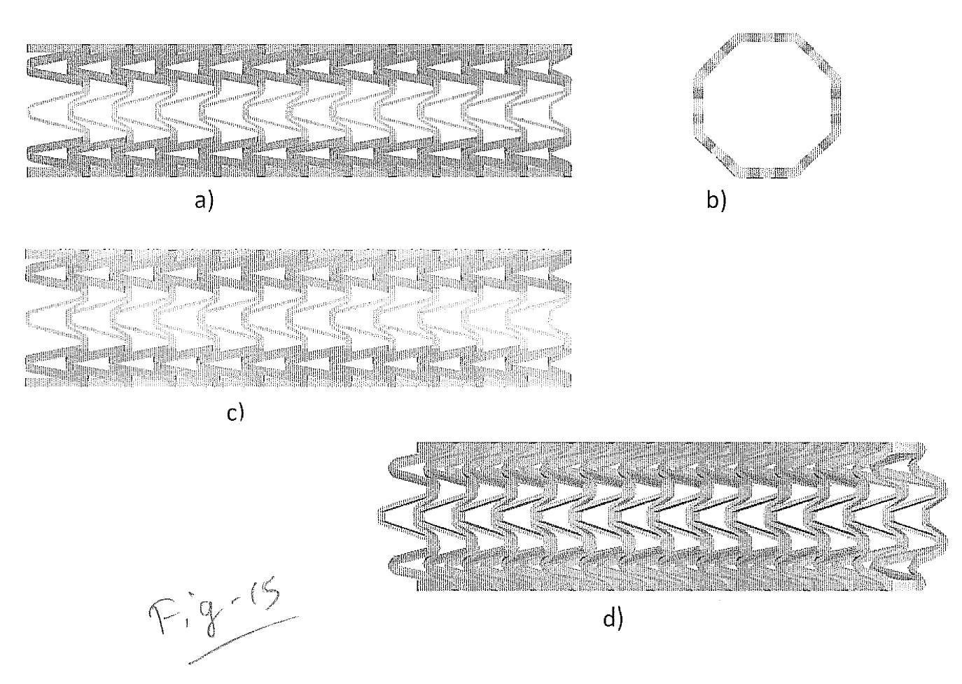

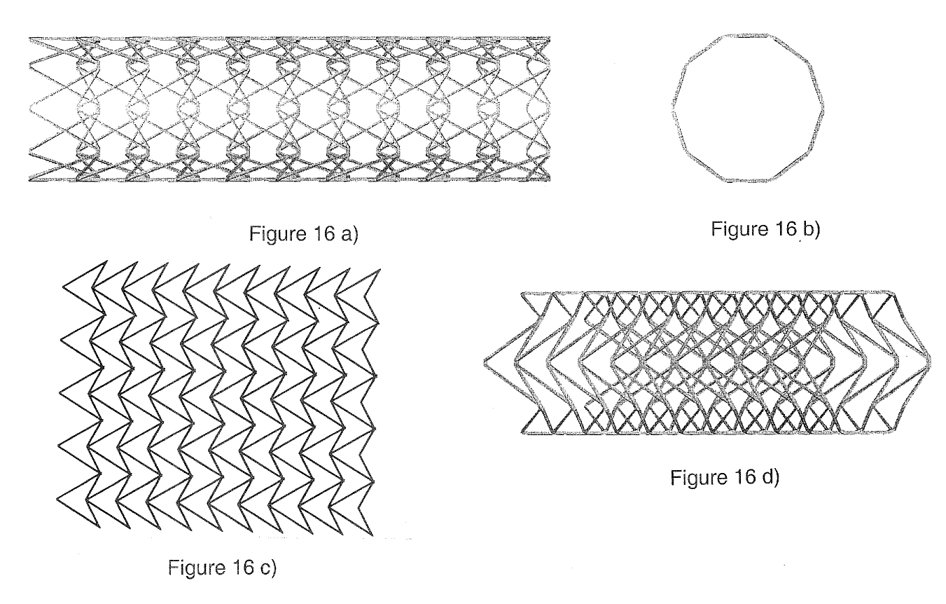

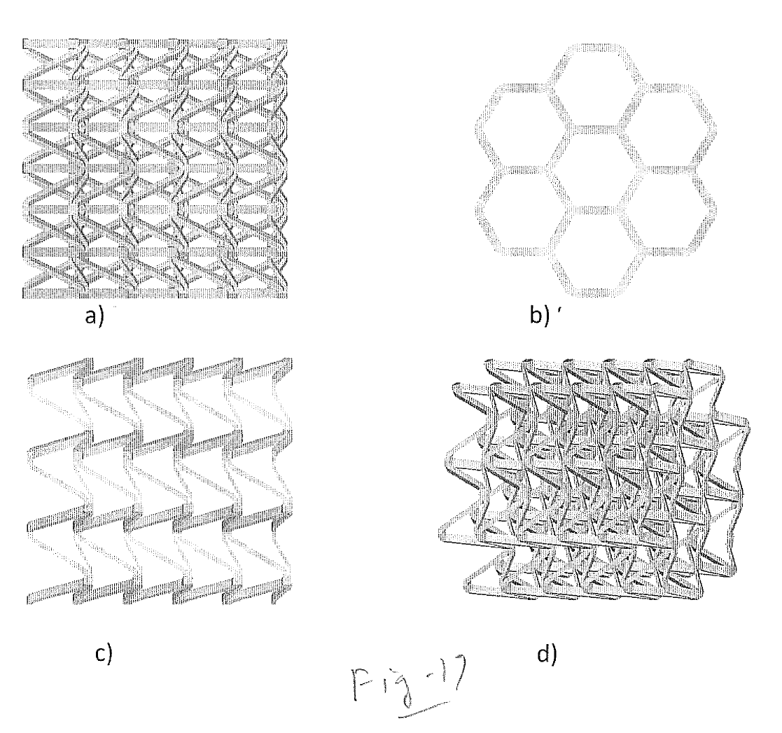

FIG. 14 is a drawing of a different auxetic stent using a V-type cell, having the octagonal cross section shown in FIG. 14B. Again, as with the design of FIG. 13, the tendon and stuffer members are bent such that four full unit cells are used peripherally around the device, resulting, however, in an eight-sided cross section. FIG. 14A is a front view; FIG. 14B is a side view; FIG. 14C shows an expanded plane view, and FIG. 14D is an isometric view. FIG. 15 illustrates a same configuration of FIG. 14 with a different thickness of the unit cells in accordance with the invention. FIG. 16 shows an octagonal (ten-sided) arrangement.

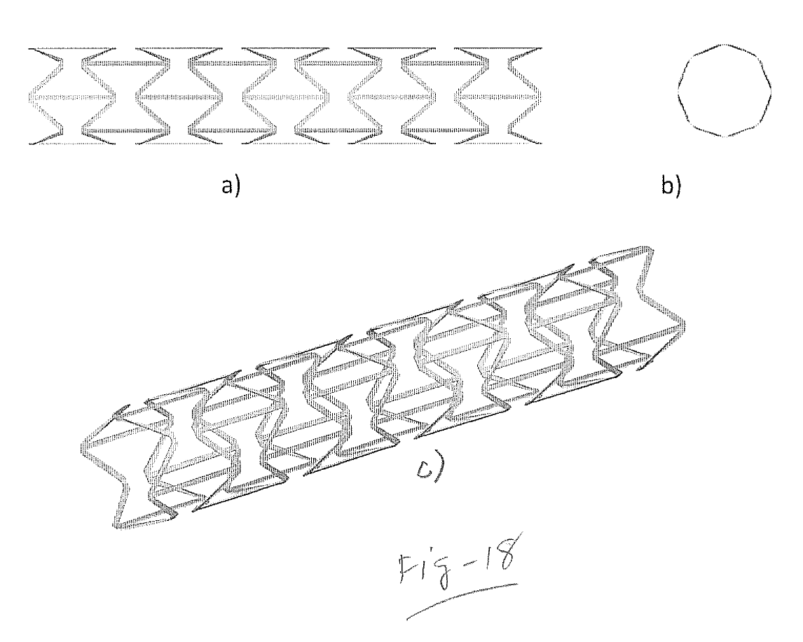

In the embodiments thus described, the channel through the finished stent is “hollow” in the sense that there are no intervening cells. However, this need not be the case, as shown in the honeycomb V-type auxetic stent of FIG. 17. As best seen in the side view of FIG. 17B, seven cells are arranged to form a honeycomb-type pattern which is repeated through the length of the stent. FIG. 17A is a front view; FIG. 17C is a top view, and FIG. 17D is an isometric view. While designs of this type fabricated in accordance with the invention replace members down the channel of the finished stent, the overall structure may be much stronger and more durable for certain applications.

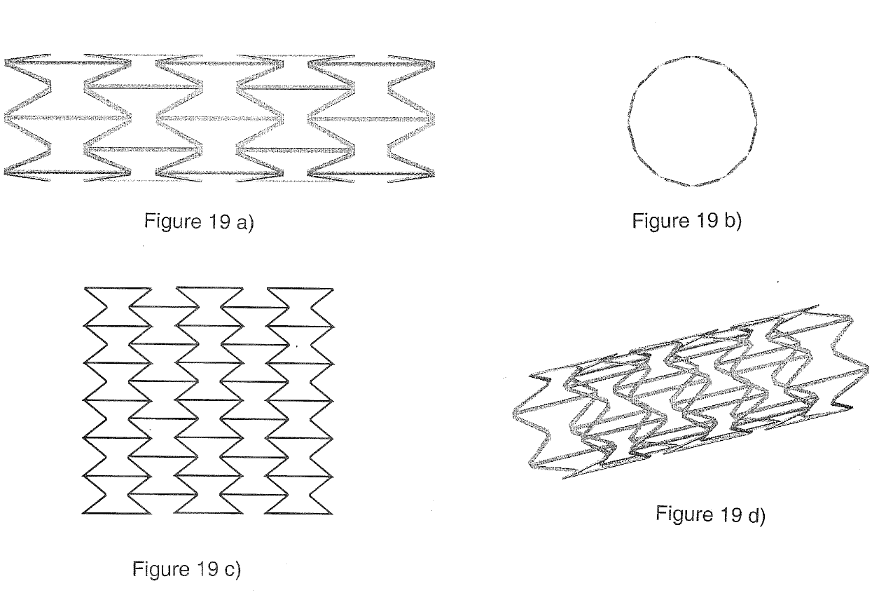

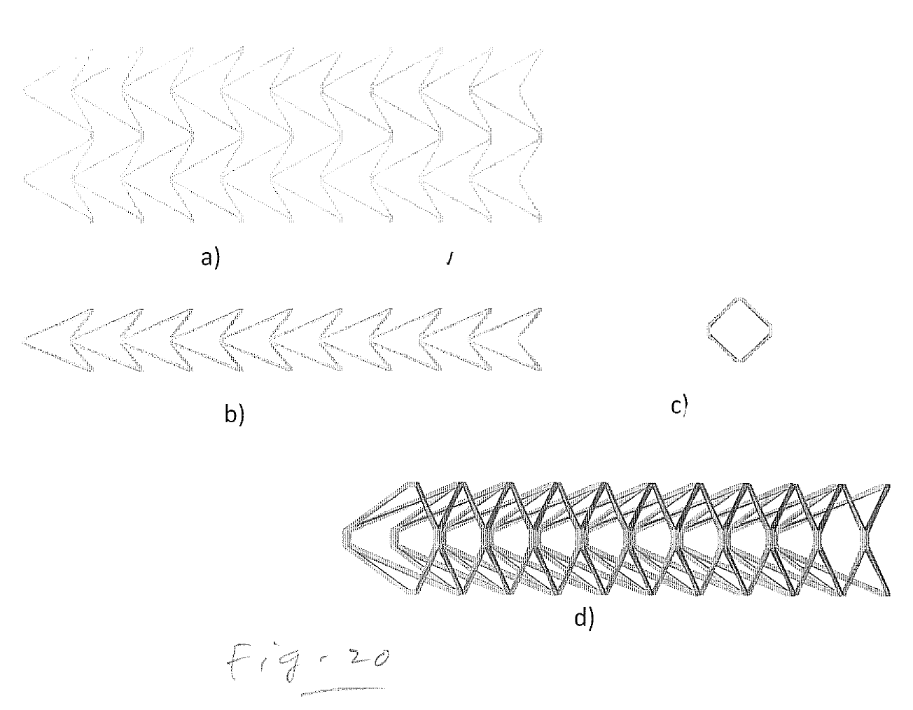

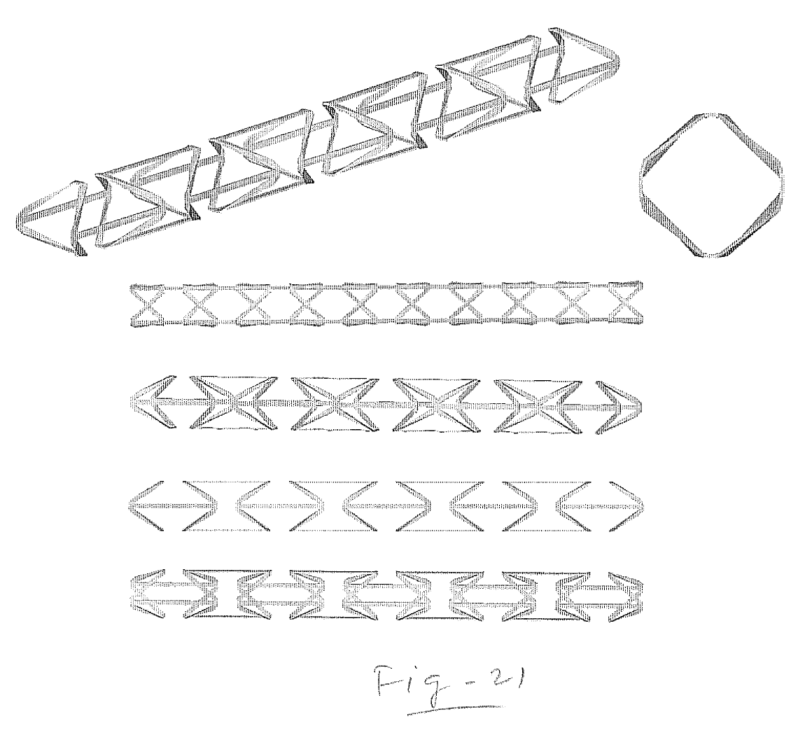

FIG. 18 is a drawing which shows a stent having an octagonal cross section (FIG. 18B) constructed from X-type unit cells. FIG. 18A is a front view, and FIG. 18C is an isometric drawing. FIG. 19 is a higher-order X-type auxetic structure, having a twelve-sided cross section, as shown in the side view of FIG. 19B. FIG. 19A is a front view; FIG. 19C is an expanded plane view; and FIG. 19D is an isometric representation. FIG. 20 illustrates a cubic V-type auxetic stent; that is, a stent constructed with the expanded plane view of FIG. 20A, having the four-sided cross section as shown in FIG. 20C. FIG. 20B is a top view, and FIG. 20D is an isometric view. FIG. 21 shows a square X-type auxetic stent constructed in accordance with the invention.

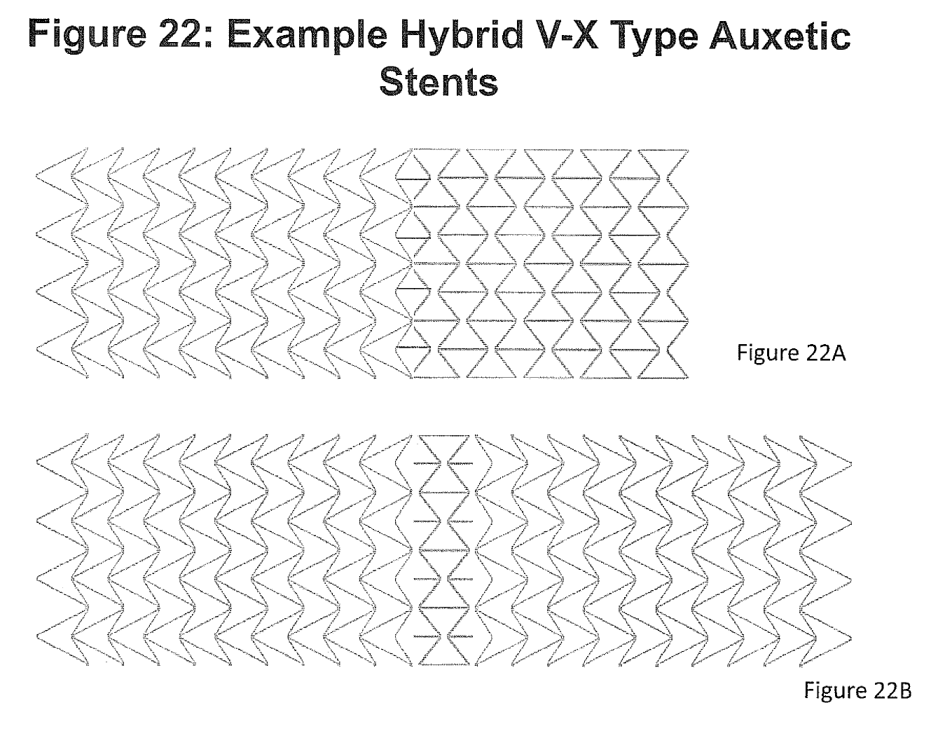

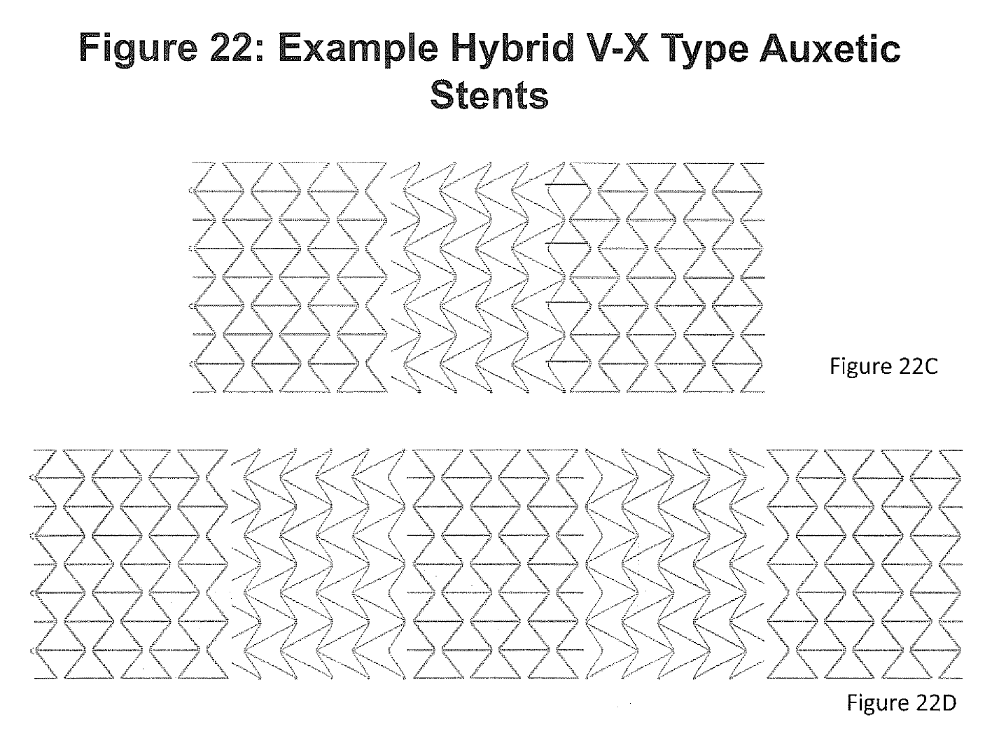

One advantage of the invention is that different types of unit cells may be combined to form a hybrid auxetic structure. Such an approach may be particularly advantageous in terms of medical/surgical stent designs in that particular portions of the length of the stent such as a section (or sections) of stent may have a larger girth and/or be more resistant to externally applied pressure. With such design goals in mind, FIG. 22A illustrates an example configuration of the hybrid stents, which combines a plurality of V-type unit cells (in the left half) and X-type unit cells (in the right half). FIG. 22B illustrates another example configuration of the hybrid stents, which combines a plurality of X-type unit cells in the middle section and V-type unit cells in the rest part of the stent. FIG. 22C illustrates the third example configuration of the hybrid stents, in which middle section is made of V-type cells and the rest part is made of X-type cells. FIG. 22D illustrates another example configuration of the hybrid stents, left, middle, and right sections are made of X-type cells and the rest part is made of V-type cells.

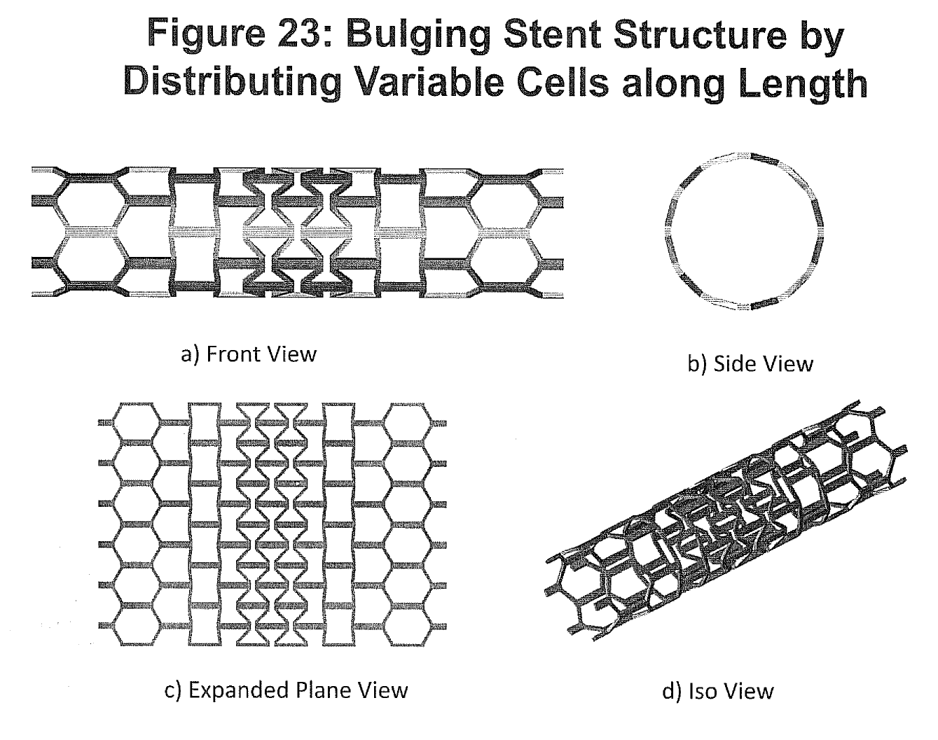

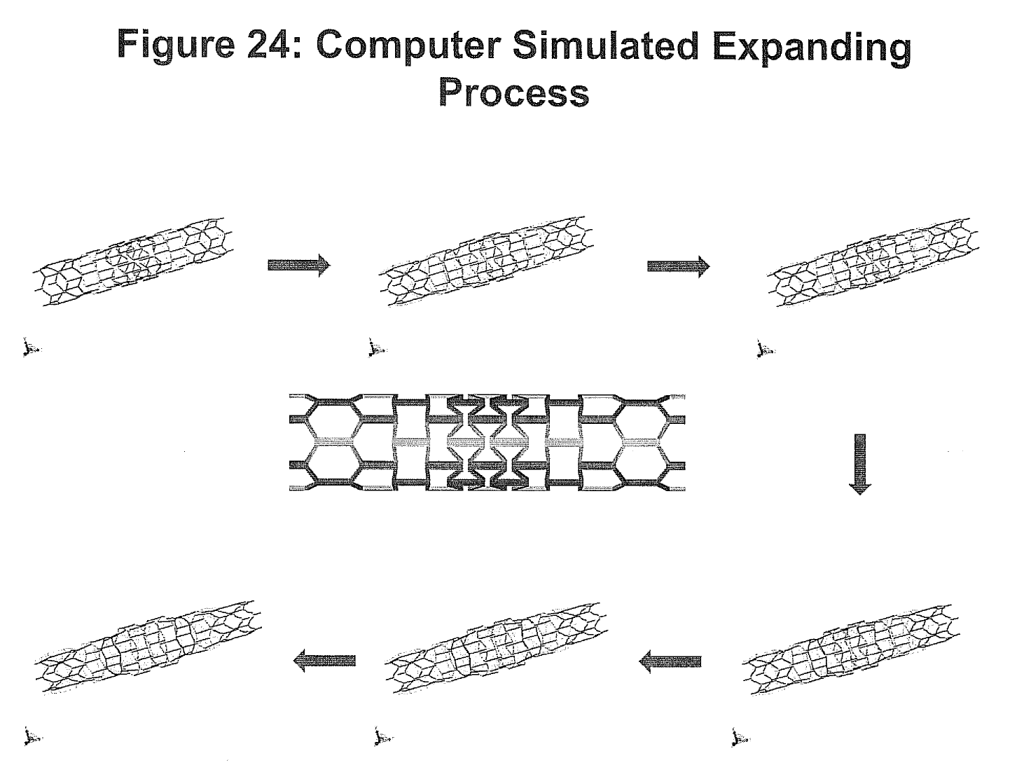

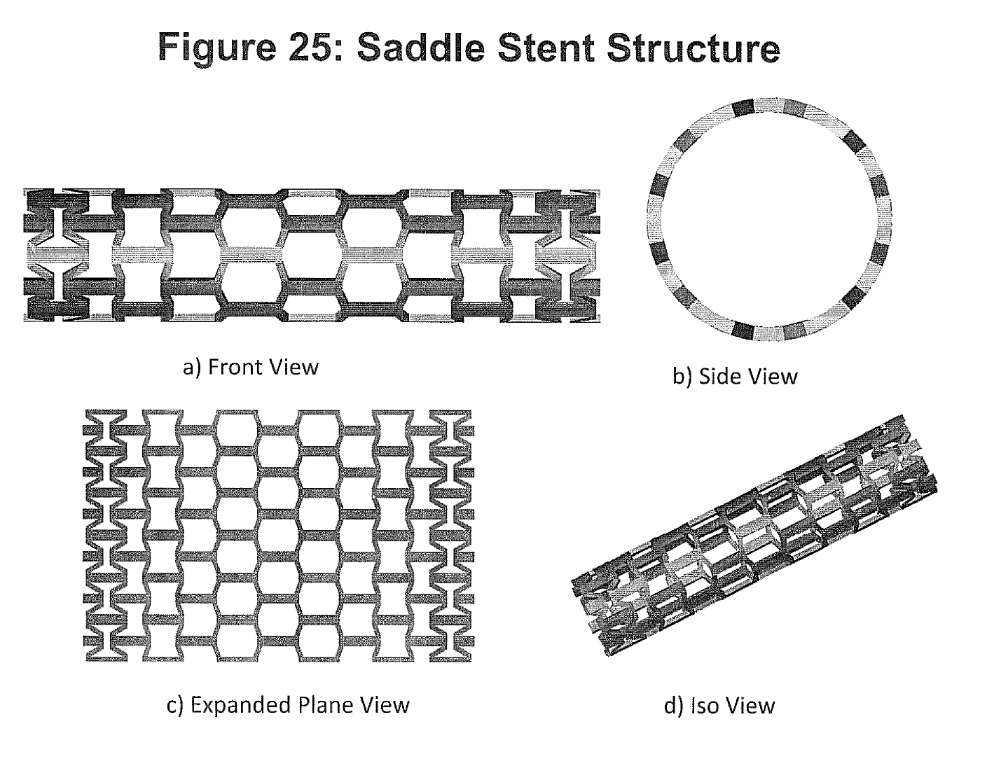

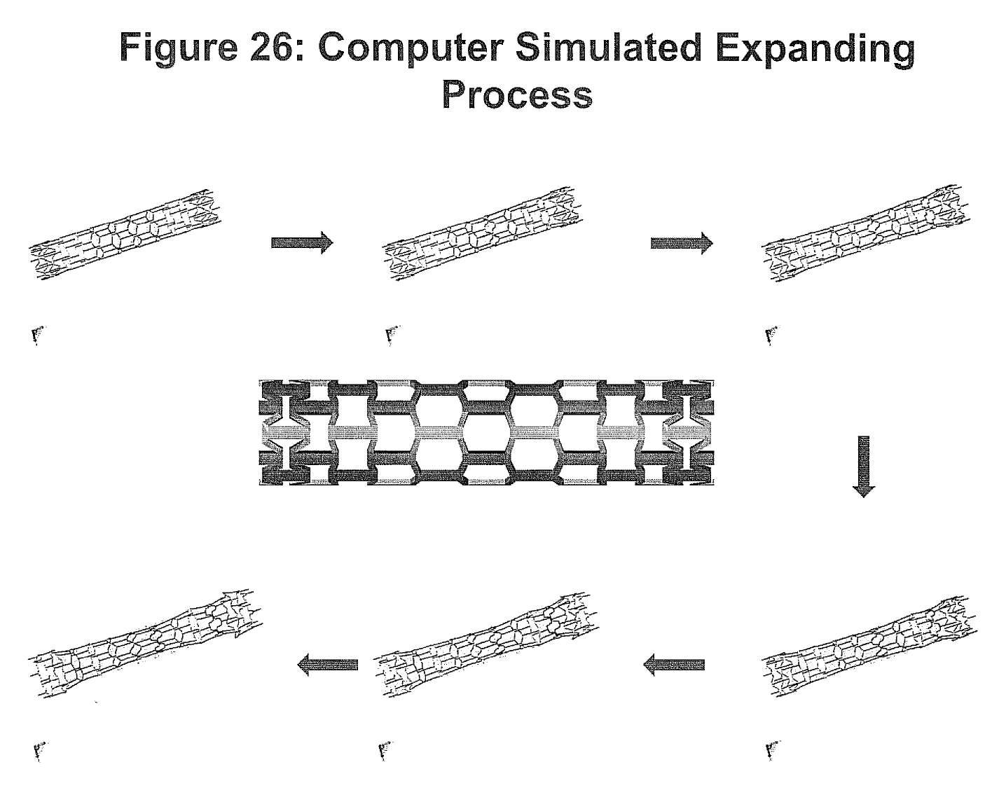

Another advantage of the invention is that the unit cells (defined in FIG. 1 and FIG. 6) can be varied cell by cell to form the stents that can deform to particularly predefined shapes. Such an approach may be particularly advantageous in terms of medical/surgical stent designs that require a special shape with predefined curvature and/or varied cross-section. FIG. 23 illustrates an example configuration of the stents designed to have a bulging effect by varying design variables, along the length of the stent, of the X-type cell defined in FIG. 6. FIG. 24 illustrates computer simulation results of the stent described in FIG. 23, which shows a bulge is formed in the middle of the stent due to the elongation of the stent under a tension force. FIG. 25 illustrates another example configuration of the stents designed to have a bulging effect at the two ends of the stent by varying the cell variables along the length of the stent. FIG. 26 illustrates computer simulation results of the stent described in FIG. 25, which shows two bulges are formed at the ends of the stent due to the elongation of the stent under a tension force. Similar variations of the stents can be obtained for the stents with the V-type cells defined in FIG. 1.

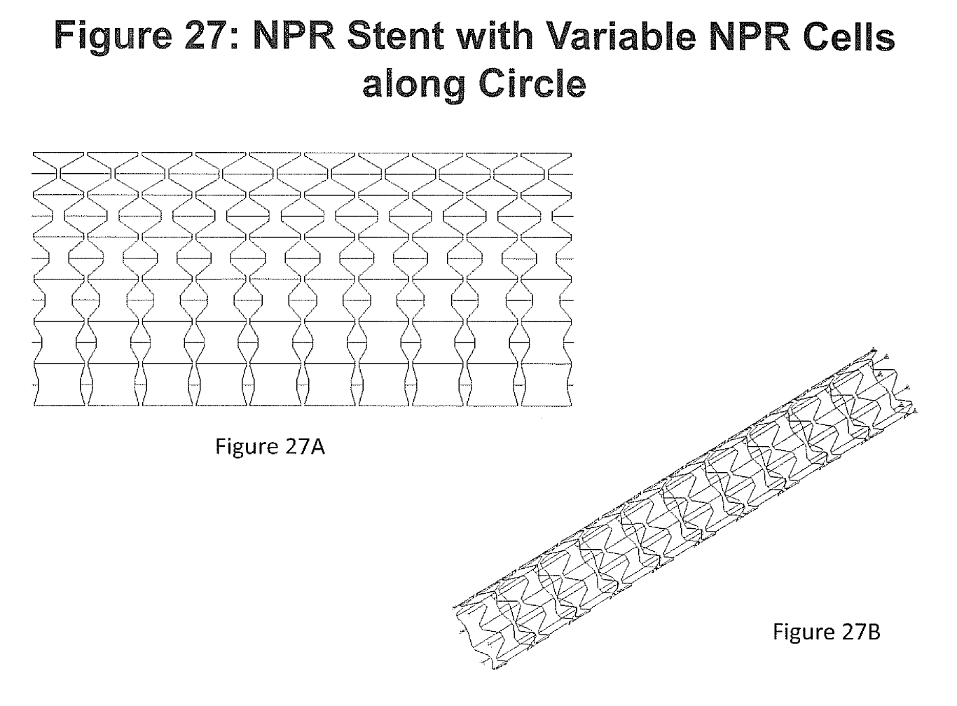

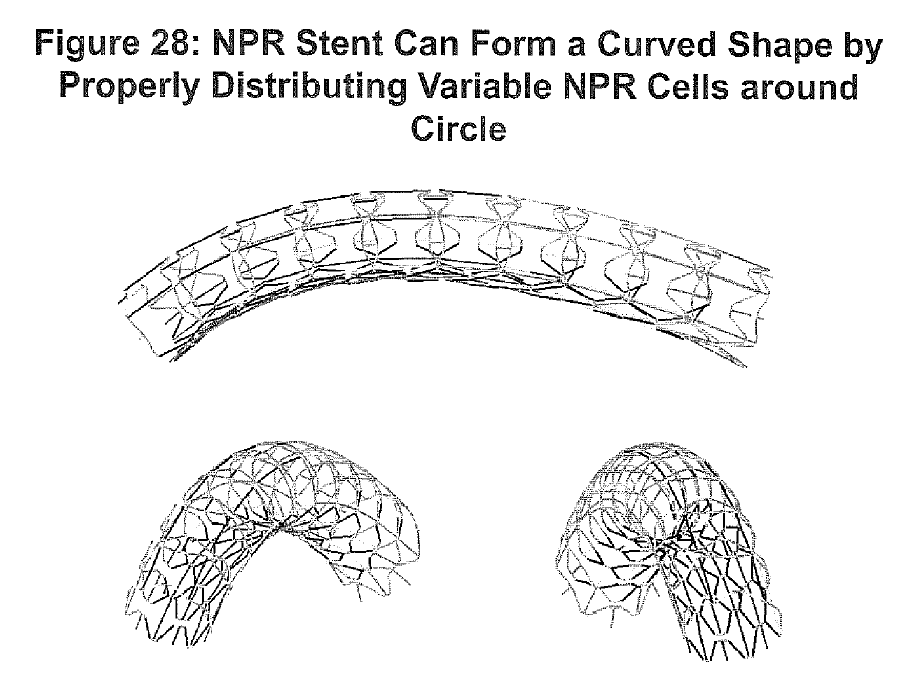

The unit cells can be varied not only along the length (axial) direction but also the circle direction to form the stents that can deform to predefined shapes such as with a curved center line along the axial direction. FIG. 27B illustrates an example configuration of the stents that can deform to a curved shape by varying the cell variables defined in FIG. 6 along the circle direction of the stent. FIG. 28 illustrates computer simulation results of the stent described in FIG. 27B, which shows the stent deformed to a curved shape due to elongation of the stent under a tension force. Similar variations of the stents can be obtained for the stents with the V-type cells defined in FIG. 1.

The stuffer and tendon members in a stent may be made of same material or different materials. An ideal stent material is fully corrosion resistant, vascular and bio-compatible, fatigue resistant, and visible using standard X-ray and MRI methodology. The stuffers and tendons may be made of biocompatible metals, which include, but not limited to, stainless steels, gold-plated hybrid materials, titanium alloys, cobalt based alloys (cobalt-chromium), tantalum and tantalum alloys, niobium, nitinol. The stuffers and tendons may also be made of biocompatible polymers, which include, but not limited to, silicone, polyethylene, polyurethane, biodegradable and bioabsorbable polymers, such as polyesters, polyorthoesters, and polyanhydrides. The stuffers and tendons may be further made of shape memory alloys or polymers, e.g. Nickel Titanium as a super-elastic shape memory alloy.

We claim:

1. An auxetic stent, comprising:
a tubular structure having two ends defining a length with a central longitudinal axis and an axial view defining a cross section;
the tubular structure being composed of a plurality of (V-type) unit cells, each unit cell comprising:
a pair of side points A and B defining a width,
a first pair of straight or curved members with constant or variable cross section interconnecting points A and B and intersecting at a point C forming a first V shape defining the “tensile” member,
a second pair of straight or curved members with constant or variable cross section interconnecting points A and B and intersecting at a point D forming a second V shape defining the “stuffer” member;
the unit cells being connected in rows with the point B of one cell being connected to point A of an adjoining cell until completing a band around the tubular structure; and
the unit cells being further connected in columns along the length of the tubular structure with the point D of one cell being connected to point C of an adjoining cell until spanning the length of the tubular structure,
whereby compression of the structure between the two ends thereof causes the cross section of the structure to shrink in size.

2. The auxetic stent of claim 1, wherein:
the members define straight segments; and
the cross section defines a regular polygon or a circle.

3. The auxetic stent of claim 1, wherein:
the members define curved segments; and
the cross section defines a regular polygon or a circle.

4. The auxetic stent of claim 1, wherein:
the members define straight or curved segments; and
the cross section defines a regular polygon or a circle, with the intersections of points of adjoining unit cells defining the vertices thereof.

5. The auxetic stent of claim 1, wherein:
the members define straight or curved segments; and
the cross section defines a square.

6. The auxetic stent of claim 1, wherein:
the members define straight or curved segments; and
the cross section defines a hexagon.

7. The auxetic stent of claim 1, wherein:
the members define straight or curved segments; and
the cross section defines an octagon.

8. The auxetic stent of claim 1, wherein:
the members define straight or curved segments; and
the cross section defines a decagon, dodecagon, or higher order of polygons.

9. The auxetic stent of claim 1, wherein:
the members define straight or curved segments; and
the cross section defines a regular polygon or a circle with constant or varied cross section dimensions along the axial direction of the stent before or after applying an axial load.

10. The auxetic stent of claim 1, wherein:
the members define straight or curved segments;
the cross section defines a regular polygon or a circle with constant or varied cross sectional dimensions along the axial direction of the stent before or after applying an axial load; and
the center line of the stent is straight or curved before or after applying the axial load.

11. The auxetic stent of claim 1, wherein:
the members define straight or curved segments;
the cross section defines a regular polygon or a circle with constant or varied cross sectional dimensions along the axial direction of the stent before or after applying an axial load; and
the center line of the stent is straight or curved before or after applying an axial load; and
the members have a constant or variable length, width, thickness, or curvature relative to the center line.

12. An auxetic stent, comprising:
a tubular structure having two ends defining a length with a central longitudinal axis and an axial view defining a cross section;
the tubular structure being composed of a plurality of (X-type) unit cells, each unit cell comprising a set of eight points interconnected with eight straight or curved members, including:
a first member interconnecting, points A and B defining a half of the left “stuffer” of the cell with a height of d₁ and width of t₁;
a second member interconnecting points B and C defining the top left “tensile” member with a length of d₂ and width of t₂;
a third member interconnecting points C and D defining the top “connecting stuffer” member with a length of d₃ and width of 2t₁;
a fourth member interconnecting points C and E defining the top right “tensile” member with a length of d₂ and width of t₂;
a fifth member interconnecting points E and F defining a half of the right “stuffer” of the cell with a height of d₁ and width of t₁;
a sixth member interconnecting points F and G defining the bottom right “tensile” member with a length of d₅ and width of t₂;
a seventh member interconnecting points G and H defining the bottom “connecting stuffer” member with a length of d₄ and width of 2t₁;
an eighth member interconnecting points G and A defining the bottom left “tensile” member with a length of d₅ and width of t₂;
wherein:
O₁ is the angle between a top tensile and the vertical line; and
O₂ is the angle between a bottom tensile and the vertical line;
the unit cells being connected in rows with the point D of one cell being connected to point H of an adjoining cell until completing a band around the tubular structure; and
the unit cells being further connected in columns along the length of the tubular structure with the line AB of one cell being connected to line EF of an adjoining cell until spanning the length of the tubular structure,
whereby compression of the structure between the two ends (D and H) thereof causes the cross section of the structure to shrink in size.

13. The auxetic stent of claim 12, wherein:
the members define straight segments; and
the cross section defines a regular polygon or a circle.

14. The auxetic stent of claim 12, wherein:
the members define curved segments; and
the cross section defines a regular polygon or a circle.

15. The auxetic stent of claim 12, wherein:
the members define straight or curved segments; and
the cross section defines a regular polygon or a circle, with the intersections of points of adjoining unit cells defining the vertices thereof.

16. The auxetic stent of claim 12, wherein:
the members define straight or curved segments; and
the cross section defines a square.

17. The auxetic stent of claim 12, wherein:
the members define straight or curved segments; and
the cross section defines a hexagon.

18. The auxetic stent of claim 12, wherein:
the members define straight or curved segments; and
the cross section defines an octagon.

19. The auxetic stent of claim 12, wherein:
the members define straight or curved segments; and
the cross section defines a decagon, dodecagon, or higher order of polygons.

20. The auxetic stent of claim 12, wherein:
the members define straight or curved segments; and
the cross section defines a regular polygon or a circle with constant or varied cross section dimensions along the axial direction of the stent before or after applying an axial load.

21. The auxetic stent of claim 12, wherein:
the members define straight or curved segments;
the cross section defines a regular polygon or a circle with constant or varied cross sectional dimensions along the axial direction of the stent before or after applying an axial load; and
the center line of the stent is straight or curved before or after applying the axial load.

22. The auxetic stent of claim 12, wherein:
the members define straight or curved segments;
the cross section defines a regular polygon or a circle with constant or varied cross sectional dimensions along the axial direction of the stent before or after applying an axial load; and
the center line of the stent is straight or curved before or after applying an axial load; and
the members have a constant or variable length, width, thickness, or curvature relative to the center line.
